(19) United States
(12) Patent Application Publication
(10) Pub. No.: US 2012/0277296 A1
(43) Pub. Date: Nov. 1, 2012

(54) Selective CB2 Receptor Agonists for Use in the Prevention or Treatment of Alcoholic Liver Disease

(76) Inventors: Sophie Lotersztajn, Creteil (FR); Fatima Teixeira-Clerc, Creteil (FR); Arlane Mallat, Creteil (FR); Alexandre Louvet, Cedex Lille (FR)

(21) Appl. No.: 13/384,802

(22) PCT Filed: Jul. 21, 2010
(86) PCT No.: PCT/EP2010/060556

§ 371 Date: Jul. 23, 2012

(30) Foreign Application Priority Data
Jul. 23, 2009 (EP) 09305700.8

(51) Int. Cl. A61K 31/352; A61P 1/16

(52) U.S. Cl. 514/454

(57) ABSTRACT

The present invention relates to selective CB2 agonists for use in the prevention or treatment of alcoholic liver disease and particularly alcoholic hepatic steatosis and liver inflammation, and pharmaceutical composition thereof.

### FIELD OF THE INVENTION

The present invention relates to the prevention or treatment of alcoholic liver disease, and particularly alcoholic hepatic steatosis and liver inflammation, using CB2 receptor agonists.

### BACKGROUND OF THE INVENTION

Alcoholic liver disease is one of the major medical complications of alcoholic abuse. Currently, alcohol accounts for the majority of liver cirrhosis in the western world and is increasingly seen in Asian countries such as Japan and India.

The three most widely recognised steps of the alcoholic liver disease are alcoholic fatty liver or alcoholic hepatic steatosis, alcoholic hepatitis and alcoholic cirrhosis. At least 80% of heavy drinkers develop steatosis, 10-35% develop alcoholic hepatitis and approximately 10% develop cirrhosis.

Alcoholic hepatic steatosis, also called alcoholic fatty liver, consists in the occupation of a large proportion of the cytoplasm of affected hepatocytes by a single large triglyceride occlusion. This state is reversible if abstinence but may progress in cirrhosis if excess alcohol intake persist.

Alcoholic hepatitis is the second main step of alcoholic liver disease and associates steatosis together with inflammation and necrosis, due to excessive intake of alcohol.

Alcoholic cirrhosis is the most severe and terminal step of the alcoholic liver disease. It is characterized by fibrosis, leading to a progressive loss of liver function. Survival for patients affected by alcoholic cirrhosis is 60%-70% at one year and 35%-50% at five years.

There is no targeted therapy for this pathology, for which abstinence is known as the most important aspect of treatment. Corticosteroids are efficient in acute forms of alcoholic hepatitis but 40% of patients fail to respond to this treatment.

So, there is a real need for new strategies of treatment, with an early patient care for better results.

Cannabinoid receptors have already been implicated in liver disorders and are seen as potential interesting targets in the pathology (Julien B et al, 2005; Teixeira-Clerc F et al, 2006; Parfienuik A et al, 2008; Deveaux V et al, 2009).

CB2 receptor has already been shown as blocking accumulation of human hepatic myofibroblasts (Julien B et al, 2005) and agonising the CB2 receptor has been proposed as a therapeutic strategy for the management of liver fibrosis (US20050143448).

On the other hand, CB2 receptors have been described as potentiating obesity-associated inflammation and complications such as insulin resistance and hepatic steatosis (Devaux V et al, 2009; WO2006138656).

It would be interesting to find new strategies of treatment for an early stage of alcoholic liver disease, knowing that the mechanisms are really distinct of those implicated in obesity-associated liver disorders.

### SUMMARY OF THE INVENTION

The present invention relates to a selective agonist of CB2 receptor for use in the prevention or treatment of alcoholic liver disease.

Particularly, the present invention relates to a selective agonist of CB2 receptor for use in the prevention or treatment of alcoholic hepatic steatosis and liver inflammation.

The present invention also relates to a pharmaceutical composition comprising a selective agonist of CB2 receptor for use in the prevention or treatment of alcoholic liver disease.

Particularly, the present invention relates to a pharmaceutical composition comprising a selective agonist of CB2 receptor for use the prevention or treatment of alcoholic hepatic steatosis and liver inflammation.

### DETAILED DESCRIPTION OF THE INVENTION

The inventors have shown that CB2 receptor activation triggers a decrease of the severity of alcoholic hepatic steatosis and the associated hepatic inflammation. Thus, the invention relates to the use of selective CB2 receptor agonists for the prevention or treatment of the early stage of alcoholic liver disease.

### DEFINITIONS

The term “receptor agonist” has its general meaning in the art and refers to a natural or synthetic compound which binds the receptor to form a receptor-agonist complex activating said receptor and receptor-agonist complex, respectively, initiating a pathway signaling and further biological processes.

The term “CB2 receptor” or “cannabinoid receptor 2”, also named Cnr2 or CX5, has its general meaning in the art (Pertwee, R G, 1999) and refers to a G-protein coupled receptor encoded by the Cnr2 gene. The term may include naturally occurring “CB2 receptor” and variants and modified forms thereof. The CB2 receptor can be from any source, but typically a mammalian (e.g., human and non-human primate) CB2 receptor, particularly a human CB2 receptor. An exemplary native human CB2 receptor amino acid sequence is provided in GenPept database under accession number NP_{—}001832 and an exemplary native human nucleotide sequence of Cnr2 mRNA is provided in GenBank database under accession number NM_{—}001841.

The term “CB2 agonist” or “CB2 receptor agonist” refers to a compound able to activate CB2 receptor, i.e. able to specifically bind the CB2 receptor and trigger various signal transducing activities of the receptor.

The term “selective CB2 agonist” or “selective CB2 receptor agonist” refers to a compound able to selectively activate CB2 receptor. In the context of the present invention, CB2 agonists are selective for the CB2 receptor as compared with the CB1 receptor. By “selective” it is meant that the affinity of the agonist for the CB2 receptor is at least 10-fold, preferably 25-fold, more preferably 100-fold and still preferably 300-fold higher than the affinity for the CB1 receptor. The affinity of an agonist for CB2 (or CB1) receptor may be quantified by measuring the activity of CB2 (or CB1) receptor in the presence a range of concentrations of said agonist in order to establish a dose-response curve. Accordingly, a selective CB2 agonist is a compound for which the ratio K_{d} CB1/K_{d} CB2 is above 10:1, preferably 25:1, more preferably 100:1, still preferably 300:1.

The agonistic activity of compounds towards the CB2 (or CB1) receptors may be determined using various methods. For example, it is known that CB1/CB2 receptors are negatively G coupled protein receptors and CB1/CB2 agonists are thus capable of inhibiting the adenylate cyclase activity. Thus, the affinity of an agonist for CB2 (and CB1) receptor may be assayed by determining the ability of said agonist to block the effect of the Forskolin (an adenylate cyclase activator) in a cAMP measurement assay. In particular, a cAMP accumulation measurement assay has been described in Rinaldi-Carmona et al. (1998) in view of Matsuda et al. (1990) and Rinaldi-Carmona et al. (1996). Typically, CHO cells stably transformed with CB1 or CB2 are grown to confluence are washed with PBS and incubated for 15 min at 37° C. in 1 ml of PBS (containing 0.25% acid-free BSA, 0.1 mM IBMX, 0.2 mM RO20-1724) in the absence or in the presence a potential selective CB2 agonist to be assayed (for instance 10⁻⁹-10⁻⁶M). Forskolin (3 μM final concentration) is added and cells are incubated for another 20 min at 37° C. The reaction is terminated by rapid aspiration of the assay medium and addition of 1.5 ml of ice-cold 50 mM Tris-HCl, pH 8, 4 mM ethylenediaminetetraacetic acid. Dishes are placed on ice for 5 min and then the extracts are transferred to a glass tube. Extracts are boiled and centrifuged for 10 min at 3500 g to eliminate cell debris. Aliquots from supernatant are dried and the cAMP concentration is determined according to any suitable method and compared in the presence or absence of the selective CB2 agonist candidate. The one skilled in the art may in particular make use of one of the many commercial kits available for cAMP measurement.

In its broadest meaning, the term “treating” or “treatment” refers to reversing, alleviating, inhibiting the progress of, or preventing the disorder or condition to which such term applies, or one or more symptoms of such disorder or condition.

In its broadest meaning, the term “preventing” or “prevention” refers to preventing the onset of a disorder in a subject. Particularly, the term “preventing an alcoholic liver disease” or “prevention of an alcoholic liver disease” refers to preventing the onset of a disorder in a subject susceptible to develop the disease, who is an alcoholic subject.

As used herein, the term “subject” denotes a mammal, such as a rodent, a feline, a canine, and a primate. Preferably, a subject according to the invention is a human.

According to the invention, the subject is a patient affected or susceptible to be affected by an alcohol liver disease, particularly at the step of alcoholic hepatic steatosis.

As used herein, the term “control” denotes a healthy subject and particularly a subject not affected by a hepatic disease, more particularly an alcoholic liver disease.

The term “biological sample” is used herein in its broadest sense. A biological sample is generally obtained from a subject. Frequently, a sample will be a “clinical sample”, i.e., a sample derived from a patient.

### Therapeutic Methods and Uses

A first object of the invention relates to a selective CB2 agonist for use in the prevention or treatment of alcoholic liver disease.

In one embodiment, the invention relates to a selective CB2 agonist for use in the prevention or treatment of alcoholic hepatic steatosis and liver inflammation.

In one embodiment, the selective CB2 agonist of the invention may be a low molecular weight antagonist, e.g. a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

Selective CB2 agonists are well known by the skilled man in the art.

A selective CB2 agonist that is contemplated by the invention is JWH-133 [(6aR,10aR)-3-(1,1-dimethylbutyl)-6a,7,10,10a-tetrahydro-6,6,9-trimethyl-6Hdibenzo[b,d]pyran] (see, e.g., Huffman et al, 1999). The structure of JWH-133 is provided here below:

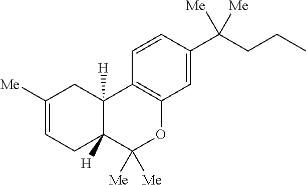

Another selective CB2 agonist that is contemplated by the invention is JWH-015 [(2-Methyl-1-propyl-1H-indol-3-yl)-1-naphthalenylmethanone] that is a drug from the aminoalkylindole family.

Another selective CB2 agonist that is contemplated by the invention is HU-308 [[(1R,2R,5R)-2-[2,6-dimethoxy-4-(2-methyloctan-2-yl)phenyl]-7,7-dimethyl-4-bicyclo[3.1.1]hept-3-enyl]methanol] is a selective CB2 agonist (L Hanus et al, 1999).

The compounds L-759,633 [(6aR,10aR)-1-methoxy-6,6,9-trimethyl-3-(2-methyloctan-2-yl)-6a,7,10,10a-tetrahydrobenzo[c]chromene] and L-759,656 [(6aR,10aR)-1-methoxy-6,6-dimethyl-9-methylidene-3-(2-methyloctan-2-yl)-7,8,10,10a-tetrahydro-6aH-benzo[c]chromene] are selective CB2 agonists that are contemplated by the invention.

Another example of selective CB2 agonist is the palmitoylethanolamide [N-(2-hydroxyethyl)hexadecanamide] (see, e.g., Facci et al, 1995).

The PCT patent application WO2005021547 also discloses CB2 agonistic compounds that can be used according to the present invention. Such compounds are described by the following general structures:

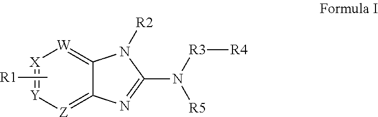

wherein
R1 is: H, C1-6alkyl, halogen, OCH3, CF3, OCF3, OCHF2, OH or C2-6alkoxy;
R2 is: C1-6 alkyl, cycloalkyl, (CH2)n-heterocycloalkyl, or (CH2)n-heteroaryl, wherein n is an integer from 1 to 3;
R3 is: CHR6, CO or SO2;
R4 is: lower alkyl, cycloalkym, heteroalkyl, heterocycloalkyl, aryl or heteroaryl;
R5 is: H or lower alkyl or heteroalkyl;
R6 is H, lower alkyl, cycloalkyl, heteroalkyl, heterocycloalkyl, aryl, heteroaryl or carboxy; and
W, X, Y and Z can be either C or N, wherein the total number of nitrogen atoms amongst W, X, Y and Z does not exceed 2.

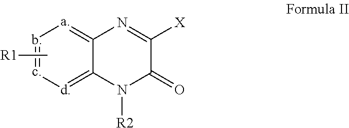

wherein
R1 is: H, C1-6alkyl, halogen, OCH3, CF3, OCF3, OCHF2, OH or C2-6alkoxy;
R2 is: C1-6alkyl, cycloalkyl, (CH2)n-heteroalkyl, (CH2)n-heterocycloalkyl or (CH2)n-heteroaryl, wherein n is an integer from 1 to 3;
X is: N(R3-R4)(R5), C(O)Y or C(NH)Y;
R3 is: CHR6, CO or SO2;
R4 is: lower alkyl, cycloalkym, heteroalkyl, heterocycloalkyl, aryl or heteroaryl;
R5 is: H or lower alkyl or heteroalkyl;
R6 is H, lower alkyl, cycloalkyl, heteroalkyl, heterocycloalkyl, aryl, heteroaryl or carboxy;
Y is: N(R3-R4)(R5) or C1-6alkyl, C1-6alkenyl, C1-6alkynyl, cycloalkyl, heteroaryl, (CH2)n-heterocycloalkyl, (CH2)n-aryl or (CH2)n-heteroaryl, wherein n is an integer of from 1 to 3; and
a., b., c. and d. can be either C or N, wherein the total number of nitrogen atoms amongst a., b., c. and d. does not exceed 2.

The US patent application number US 2005165118 discloses pinene derivatives that can also be used. Such compounds have the following formula:

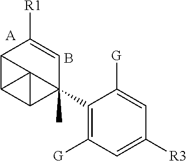

wherein: A---B designates an optional double bond, R1 is —CH2OH; G is hydrogen or —OR2 wherein R2 is a lower alkyl group; and R3 is a straight or branched chain C5-C12 alkyl.

Other selective CB2 receptor agonists that can be used include those disclosed in published US patent application US2004034090, entitled “3-Arylindole derivatives and their use as CB2 receptor agonists”. Agonists of this class have the following general structure:

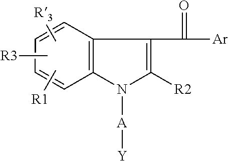

in which:
Ar represents:
   a) a phenyl mono-, di- or trisubstituted by one or more groups chosen from: a halogen atom, a (C1-C4)alkyl, a trifluoromethyl, an amino, a nitro, a hydroxyl, a (C1-C4)alkoxy, a (C1-C4)alkylsulphanyl or a (C1-C4)alkylsulphonyl;
   b) a naphthyl which is unsubstituted or substituted once or twice by a halogen atom, a (C1-C4)alkyl or a trifluoromethyl;
A represents a C2-C6 alkylene radical;
Y represents a group chosen from SR4, SOR4, SO2R4, SO2NR5R6, N(R7)SO2R4, OR4 or NR7SO2NR5R6;
R1, R3 and R′3 represent, each independently of one another, hydrogen, a hydroxyl, a halogen atom, a (C1-C4)alkyl, a trifluoromethyl or a (C1-C4)alkoxy;
R2 represents hydrogen or a (C1-C4)alkyl;
R4 represents a (C1-C4)alkyl or a trifluoromethyl
R5 and R6 each independently represent hydrogen or a (C1-C4)alkyl; and
R7 represents hydrogen or a (C1-C4)alkyl.

Still other classes of selective CB2 agonists that are contemplated by the invention include but are not limited to, those described in U.S. Pat. Nos. 7,214,716; 6,013,648 and 5,605,906; published PCT applications WO02085866, WO0132169, WO0128497, WO9700860 and WO9618391; 3) published U.S. Patent Applications US2004077643 and US2002173528; and EP patent applications EP1306373 and EP1374903, that are incorporated herein by reference in their entirety.

Selective CB2 agonists can also be biological molecules.

In a particular embodiment, such a molecule could be an antibody directed against the CB2 receptor, which is able to promote or enhance the CB2 activity.

Antibodies can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see, e.g., U.S. Pat. No. 4,946,778) can be adapted to produce anti-CB2 receptor single chain antibodies. Antibodies that are contemplated by the invention include antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to CB2 receptor.

Humanized antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397). According to the invention, such antibodies directed against CB2 receptor will be assayed for their CB2 agonistic activity by several methods well known in the art such as those described above.

In another embodiment the blocking agent of the purinergic signalling pathway is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

According to the invention, such antibodies and aptamer directed against CB2 receptor will be assayed for their CB2 agonistic activity by several methods well known in the art such as those described above.

A further object of the invention relates to methods and compositions for prevention or treatment of a subject affected by alcohol liver disease.

In one embodiment, the invention relates to a method for prevention or treatment of a subject affected by alcohol liver disease comprising a step of administering a selective CB2 agonist as above described.

In a particular embodiment, the invention relates to a method for prevention or treatment of a subject affected by alcoholic hepatic steatosis or liver inflammation comprising a step of administering a selective CB2 agonist.

The CB2 receptor agonist may be administered in the form of a pharmaceutical composition. Preferably, said compound is administered in a therapeutically effective amount.

By a “therapeutically effective amount” is meant a sufficient amount of the CB2 receptor agonist to prevent or treat alcoholic liver disease at a reasonable benefit/risk ratio applicable to any medical treatment.

It is understood that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, gender and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved.

### Pharmaceutical Compositions

A further object of the invention is a pharmaceutical composition comprising a selective CB2 agonist for use in the prevention or treatment of alcoholic liver disease.

In a particular embodiment, the invention relates to a pharmaceutical composition comprising a selective CB2 agonist for use in the prevention or treatment of alcoholic hepatic steatosis and liver inflammation.

The CB2 receptor agonist may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

According to the invention, the CB2 receptor agonists can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The CB2 receptor agonist of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

### Screening Methods

Selective CB2 receptor agonists of the invention can be further identified by screening methods described in the state of the art. The screening methods of the invention can be carried out according to known methods.

The screening method may measure the binding of a candidate compound to CB2 receptor, or to cells or membranes bearing CB2 receptor, or a fusion protein thereof by means of a label directly or indirectly associated with the candidate compound. Alternatively, a screening method may involve measuring or, qualitatively or quantitatively, detecting the competition of binding of a candidate compound to the receptor with a labelled competitor (e.g., substrate).

For example, CB2 receptor cDNA may be inserted into an expression vector that contains necessary elements for the transcription and translation of the inserted coding sequence. Following vector/host systems may be utilized such as Baculovirus/Sf9 Insect Cells Retrovirus/Mammalian cell lines like HepB3, LLC-PK1, MDCKII, CHO, HEK293 Expression vector/Mammalian cell lines like HepB3, LLC-PK1, MDCKII, CHO, HEK293. Such vectors may be then used to transfect cells so that said cells express recombinant CB2 receptor at their membrane. It is also possible to use cell lines expressing endogenous CB2 protein, such as RAW264.7

Selective CB2 agonist candidates can be tested by assaying their CB2 agonistic activity by several methods well known in the art such as those described above.

### Diagnostics Methods of the Invention

A further aspect of the invention relates to a method of testing a subject thought to have or be predisposed to having an alcoholic liver disease, which comprises the step of analyzing a biological sample from said subject for:

(i) detecting the presence of a mutation in the gene Cnr2 encoding for the CB2 receptor and/or its associated promoter, and/or

(ii) analyzing the expression of the Cnr2 gene.

Typical techniques for detecting a mutation in the Cnr2 gene may include restriction fragment length polymorphism, hybridisation techniques, DNA sequencing, exonuclease resistance, microsequencing, solid phase extension using ddNTPs, extension in solution using ddNTPs, oligonucleotide assays, methods for detecting single nucleotide polymorphism such as dynamic allele-specific hybridisation, ligation chain reaction, mini-sequencing, DNA “chips”, allele-specific oligonucleotide hybridisation with single or dual-labelled probes merged with PCR or with molecular beacons, and others.

Analyzing the expression of the Cnr2 gene may be assessed by any of a wide variety of well-known methods for detecting expression of a transcribed nucleic acid or translated protein.

In a preferred embodiment, the expression of the Cnr2 gene is assessed by analyzing the expression of mRNA transcript or mRNA precursors, such as nascent RNA, of said gene. Said analysis can be assessed by preparing mRNA/cDNA from cells in a biological sample from a subject, and hybridizing the mRNA/cDNA with a reference polynucleotide. The prepared mRNA/cDNA can be used in hybridization or amplification assays that include, but are not limited to, Southern or Northern analyses, polymerase chain reaction analyses, such as quantitative PCR (TaqMan), and probes arrays such as GeneChip™ DNA Arrays (AFFYMETRIX).

Advantageously, the analysis of the expression level of mRNA transcribed from the Cnr2 gene involves the process of nucleic acid amplification, e.g., by RT-PCR (the experimental embodiment set forth in U.S. Pat. No. 4,683,202), ligase chain reaction (Barany, 1991), self sustained sequence replication (Guatelli et al., 1990), transcriptional amplification system (Kwoh et al., 1989), Q-Beta Replicase (Lizardi et al., 1988), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

In another preferred embodiment, the expression of the Cnr2 gene is assessed by analyzing the expression of the protein translated from said gene. Said analysis can be assessed using an antibody (e.g., a radio-labeled, chromophore-labeled, fluorophore-labeled, or enzyme-labeled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the CB2 receptor.

Said analysis can be assessed by a variety of techniques well known from one of skill in the art including, but not limited to, enzyme immunoassay (EIA), radioimmunoassay (RIA), Western blot analysis and enzyme linked immunoabsorbant assay (RIA).

The method of the invention may comprise comparing the level of expression of the Cnr2 gene in a biological sample from a subject with the normal expression level of said gene in a control. A significantly different level of expression of said gene in the biological sample of a subject as compared to the normal expression level is an indication that the patient has or is predisposed to developing an alcoholic liver disease. The “normal” level of expression of the Cnr2 gene is the level of expression of said gene in a biological sample of a subject not afflicted by any hepatic disease. Preferably, said normal level of expression is assessed in a control sample (e.g., sample from a healthy subject, which is not afflicted by any disease associated with an increased retinal vascular permeability) and preferably, the average expression level of said gene in several control samples.

The invention will be further illustrated by the following figures and examples. However, these examples and figures should not be interpreted in any way as limiting the scope of the present invention.

### FIGURES

FIG. 1: Impact of CB2 receptor on hepatic steatosis in response to chronic ethanol feeding. WT and Cnr2−/− mice were fed a modified Lieber-De Carli ethanol diet or a control diet for 10 days. A-C: Genetic invalidation of the CB2 receptor worsens hepatic steatosis A. Liver to body weight ratio B: Mean steatosis score C: Hepatic triglyceride content D: WT mice received a daily intraperitoneal injection of the CB2 agonist JWH-133 (3 mg/kg) or vehicle during the 10 day course of ethanol feeding. Treatment with JWH-133 is associated with a decrease in liver/body weight ratio and in triglyceride content. †p<0.05 for WT mice fed a control diet vs. WT mice fed EtOH, *p<0.05 for CB2−/− mice fed EtOH vs. WT mice fed EtOH. ¤p<0.05 for JWH-133 vs. vehicle in WT mice fed EtOH.

FIG. 2: Impact of CB2 receptor on hepatic inflammation in response to chronic ethanol feeding. A-C: Genetic invalidation of the CB2 receptor enhances hepatic inflammation in response to ethanol feeding A: Inflammation score B: Myeloperoxidase activity revealing the presence of PMN cells in ethanol-fed mice C: TNF mRNA expression. D: WT mice received a daily intraperitoneal injection of the CB2 agonist JWH-133 (3 mg/kg) or vehicle during the 10 day course of ethanol feeding. Treatment with JWH-133 prevents the increase in TNF-α mRNA expression elicited by chronic ethanol feeding. †p<0.05 for WT mice fed a control diet vs. WT mice fed EtOH, *p<0.05 for CB2−/− mice fed EtOH vs. WT mice fed EtOH ¤p<0.05 for JWH-133 vs. vehicle in WT mice fed EtOH.

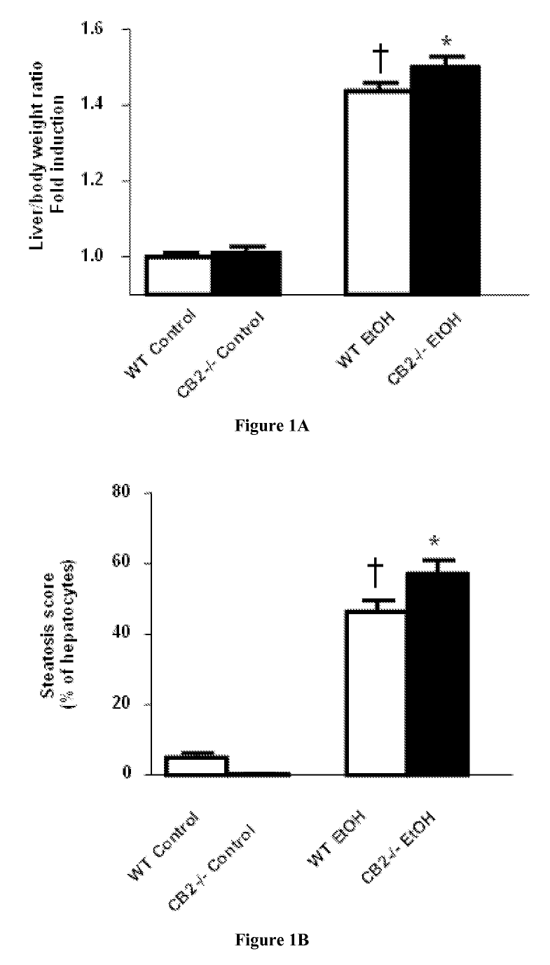

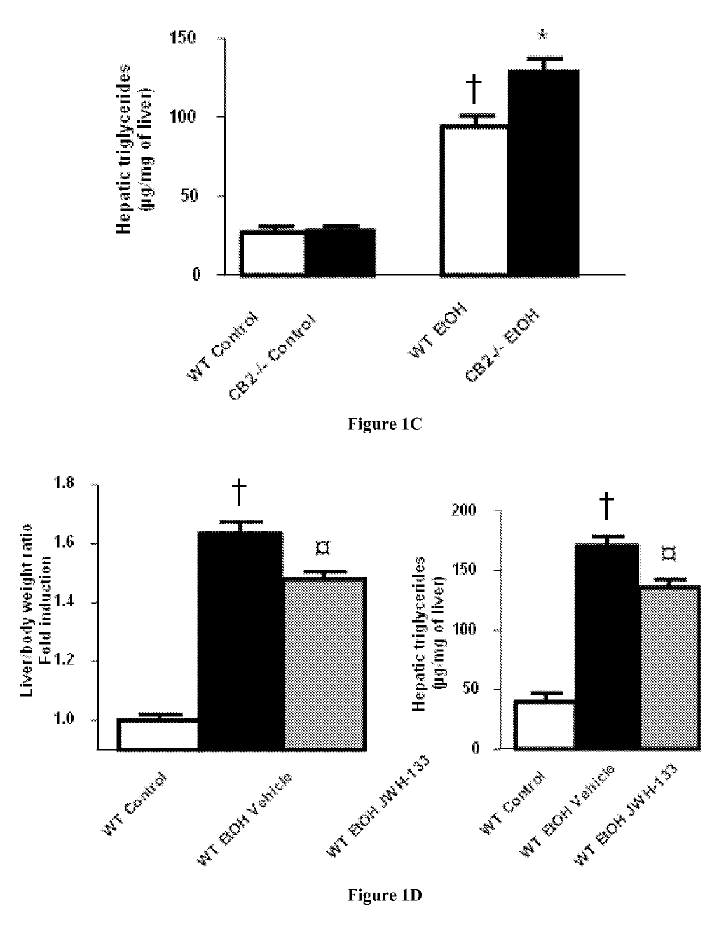

### EXAMPLE

### Material & Methods

Materials.

Ingredients for the Lieber de Carli diet were from MPBiomedicals (Illkirch, France). The CB2 agonist JWH-133 was obtained from Tocris (ThermoFisher, Illkirch, France). Absolute ethanol was obtained from Carlo Erba Reactifs, Val-de-Reuil, France.

Animals and Experimental Design.

Animals. Female mice (8-10 week old) were used for the experiments and included wild type (WT) C57B1/6J mice (Janvier, France) and mice with a targeted mutation of the Cnr2 gene (Buckley et al. 2000). Homozygous Cnr2−/− animals were obtained from heterozygous Cnr2+/− mice that were backcrossed with WT C57Bl/6J animals over 10 generations, and further intercrossed to obtain homozygous animals. Genotyping was performed on tail genomic DNA using QuantiTect™ SYBR® Green PCR kit (Qiagen). Animals were housed in temperature and humidity controlled rooms, kept on a 12-h light/dark cycle and provided unrestricted amounts of food and water. Animal procedures were conducted in accordance with French government policies (Services Vétérinaires de la Santé et de la Production Animale, Ministère de l'Agriculture).

Chronic exposure to ethanol. WT mice and Cnr2−/− mice were fed a liquid diet adapted from the Lieber-De Carli classical regimen; the liquid diet was prepared according to the composition given in Gustot et al. (Gustot et al, 2006). Mice were randomized into ethanol fed and pair fed groups and then adapted to control liquid diet ad libitum for 7 days. The ethanol fed group was allowed free access to a 6.3% vol/vol ethanol diet. Control mice were pair-fed with diets that isocalorically substituted dextrin-maltose for ethanol over the entire feeding period. Body weight and food intake were measured daily.

The impact of the CB2 agonist JWH-133 or its vehicle was evaluated in WT mice that were treated with a daily intraperitoneal injection of JWH-133 (3 mg/kg) or its vehicle during the 10 day feeding with ethanol. JWH-133 was freshly dissolved each day in a vehicle solution containing 1 drop of Tween 20 in 0.1 ml dimethylsulfoxide (DMSO), sonicated, and further diluted 50 times in NaCl 9%. Body weight and food intake were measured daily.

All the experiments included at least 5 control-fed mice and 15 ethanol-fed animals. Mice were sacrificed after overnight fasting. The liver was removed, weighed and either fixed in buffered formalin, or snap frozen in liquid nitrogen. All samples were stored at −80° C. until use.

Serum analysis. Blood was collected at the time of sacrifice, or 6 hours before fasting for determination of serum ethanol levels. Transaminase activity (alanine aminotransferase (ALT) and aspartate aminotransferase (AST) activities were measured on an automated analyzer in the Biochemistry Department of the Henri Mondor Hospital and serum ethanol levels by liquid phase chromatography.

Hepatic triglyceride quantification. Hepatic triglycerides were extracted from 50 mg of liver homogenates by homogenization in 1 ml of chloroform-methanol (2:1 v/v) using TissueLyser (Qiagen). The homogenate was centrifuged for 10 min at 1000 g, and the lipid phase was diluted with 0.2 ml water. Following an additional centrifugation for 20 min at 2400 rpm, the lipid extract was collected from the lower phase, evaporated and dissolved in 1 ml of 2-propanol. Triglycerides were quantified with a triglyceride determination kit (Sigma), on liver samples from 10-15 animals per group.

RNA preparation and RT-PCR. Total RNA was extracted using RNeasy® Lipid Tissue Mini kit (Qiagen). Quantitative real time-PCR was carried out on a Light Cycler (Roche Diagnostics), as previously described (Julien et al. 2005). Oligonucleotide primers (MWG Biotech) for the following mouse genes were used: mouse Rn18S; mouse Tnf, PCR amplified products were analyzed on a 2% agarose gel and sequenced.

Liver histology. Liver specimens were fixed in 10% formalin, paraffin-embedded tissue sections (4 μm) were stained with hematoxylin-eosin for routine examination. Steatosis and inflammation were blindly assessed on 4 random fragments from different areas of each liver (n=30 WT and n=27 Cnr2−/− ethanol-fed animals). Steatosis was staged according to the percentage of hepatocytes containing cytoplasmic vacuoles. Inflammation was staged on a scale of 0 to 3 according to the intensity of the inflammatory infiltrate: 0 (absence), 1 (moderate), 2 (mild), 3 (severe).

Immunohistochemistry. Immunohistochemical detection of myeloperoxydase was performed in liver formalin-fixed, parraffin-embedded tissue sections (4 μm), using antibody against myeloperoxydase (1/750, Dako) and biotinylated secondary antibody (1/100, Santa Cruz Biotechnology). The signal was amplified with phosphatase alkaline-conjugated streptavidin (1/20, Serotec), and alkaline phosphatase activity was revealed using fast red substrate-chromogen system (Dako). The total number of myeloperoxydase expressing cells was quantified on 10 random fields in 11 ethanol fed WT mice, and 13 ethanol fed Cnr2^{-/-} animals. Results are expressed as the number of myeloperoxydase stained cells in 10 fields.

Statistics. Results are expressed as mean±SEM and were analyzed by either Mann and Whitney test, two way ANOVA as appropriate, using PRISM 4.0 software. P<0.05 was taken as the minimum level of significance.

Results

We investigated the impact of CB2 receptor in the development of hepatic steatosis and associated inflammation in a model of chronic ethanol feeding. EtOH consumption was similar in WT and Cnr2−/− mice, and accordingly, there was no difference in serum EtOH levels between the two groups following 10 days of EtOH feeding. As previously reported by Gustot et al, 2006, both EtOH and control fed mice lost weight during the first 5 days of feeding (Table 1). However, there was no difference in body weight between WT and Cnr2−/− mice, fed either a control or an EtOH diet (Table 1).

TABLE 1

| | WT mice | Cnr2 −/− mice | | WT mice | Cnr2 −/− mice | |
|---|---|---|---|---|---|---|
| | Control diet | Control diet | p-value | EtOH diet | EtOH diet | p-value |
| Body weight before | 21.6 ± 0.3 | 21.8 ± 0.1 | 0.8 | 21.4 ± 0.2* | 21.7 ± 0.3* | 0.2 |
| EtOH administration (g) | | | | | | |
| Body weight after 10 | 21 ± 0.4 | 21.5 ± 0.1 | 0.4 | 20.8 ± 0.3* | 20.8 ± 0.4* | 0.9 |
| days of EtOH (g) | | | | | | |
| EtOH consumption | — | — | — | 18.1 ± 0.39 | 17 ± 0.29 | 0.1 |
| (g/kg/day) | | | | | | |
| Serum EtOH level (g/l) | — | — | — | 1.36 ± 0.16 | 1.19 ± 0.19 | 0.2 |
| AST (IU/l) | 92 ± 12 | 94 ± 17 | 0.9 | 209 ± 16* | 218 ± 35* | 0.3 |
| ALT (IU/l) | 31 ± 6 | 39 ± 14 | 0.8 | 84 ± 13* | 94 ± 20* | 0.7 |

(*p < 0.05 as compared to control diet fed mice).

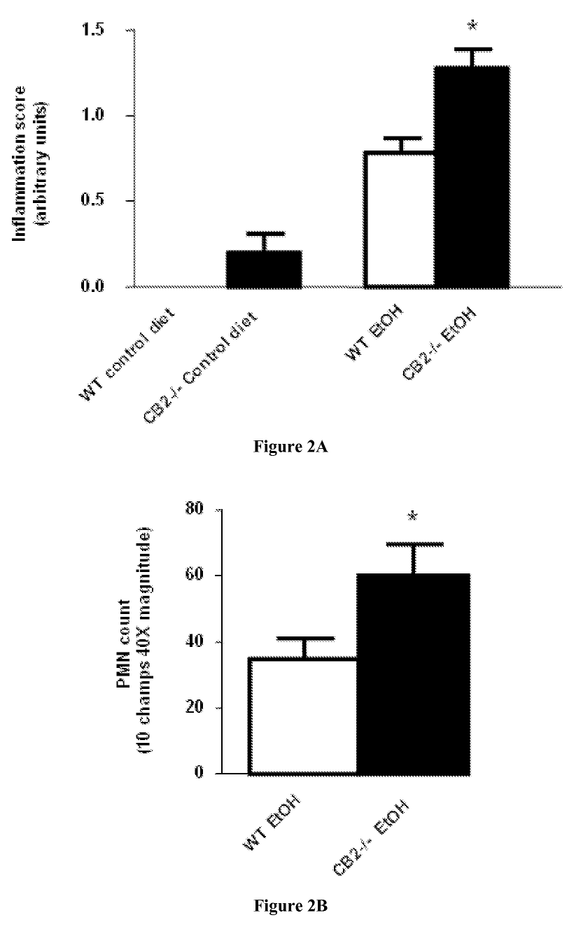

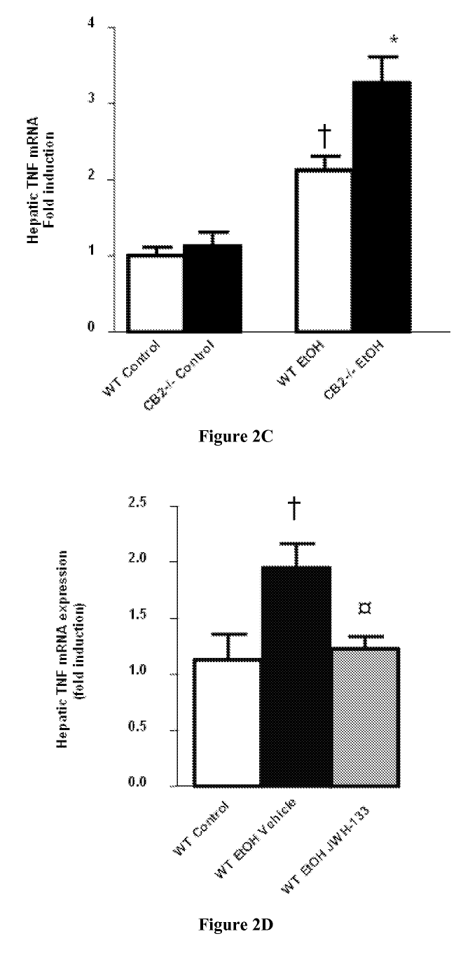

As expected, EtOH-fed WT mice exhibited a significant increase in liver/body weight ratio (FIG. 2A), associated with enhanced liver steatosis (FIGS. 2B and C), as illustrated by the increase in steatosis score and parallel enhancement of hepatic triglyceride content. Cnr2−/− mice developed a more pronounced hepatomegaly and showed a more severe steatosis, characterized by an increase in the steatosis score and hepatic triglyceride levels, as compared to their WT counterparts fed EtOH. These results demonstrated that CB2 receptor invalidation worsens hepatic steatosis promoted by chronic EtOH feeding. Conversely, the CB2 agonist JWH-133 strongly reduced liver/body weight ratio and hepatic triglyceride accumulation (FIG. 2D). Altogether, these data demonstrate that CB2 receptor activation protects the liver from EtOH-induced hepatic steatosis.

We next investigated the impact of CB2 receptor on liver inflammation. Liver samples from EtOH-fed Cnr2−/− mice showed a marked inflammatory score, whereas inflammatory infiltration was minimal in EtOH-fed WT mice (FIG. 3A). The density of polymorphonuclear cells was higher in CB2−/− mice fed ethanol as compared to their WT counterparts (FIG. 3B). However, despite an increase in transaminase levels following EtOH feeding, there was no difference between WT and Cnr2−/− mice fed EtOH (Table). Liver inflammation was also monitored by the expression of TNF-α, a cytokine that plays a central role in the pathogenesis of alcoholic liver disease. TNF-α mRNA expression was markedly increased in Cnr2−/− mice fed EtOH, as compared to WT counterparts (FIG. 3C). Conversely, administration of JWH-133 reduced TNF-α mRNA levels to control levels (FIG. 3D).

Taken together, these results demonstrate that CB2 receptors play a beneficial role in alcoholic liver disease by reducing hepatic steatosis and liver inflammation.

### REFERENCES

Throughout this application, various references describe the state of the art to which this invention pertains. The disclosures of these references are hereby incorporated by reference into the present disclosure.
Barany, Proc. Natl. Acad. Sci. USA, vol. 88, p: 189-193, 1991.
Buckley N E, McCoy K L, Mezey E, Bonner T, Zimmer A, et al. (2000) Immunomodulation by cannabinoids is absent in mice deficient for the cannabinoid CB(2) receptor. Eur J Pharmacol 396: 141-149.
Deveaux V, Cadoudal T, Ichigotani Y, Teixeira-Clerc F, Louvet A, Manin S, Nhieu J T, Belot M P, Zimmer A, Even P, Cani P D, Knauf C, Burcelin R, Bertola A, Le Marchand-Brustel Y, Gual P, Mallat A, Lotersztajn S. Cannabinoid CB2 receptor potentiates obesity-associated inflammation, insulin resistance and hepatic steatosis. PLoS One. 2009 Jun. 9; 4(6):e5844.
Facci L, Dal Toso R, Romanello S, Buriani A, Skaper S D, Leon A. Mast cells express a peripheral cannabinoid receptor with differential sensitivity to anandamide and palmitoylethanolamide. Proc Natl Acad Sci USA. 1995 Apr. 11; 92(8):3376-80.
Guatelli et al., Proc. Natl. Acad. Sci. USA, vol. 57, p: 1874-1878, 1990.
Gustot, T., Lemmers, A., Moreno, C., Nagy, N., Quertinmont, E., Nicaise, C., Franchimont, D. Louis, H. Deviere, J. Le Moine, 0. (2006) Differential liver sensitization to toll-like receptor pathways in mice with alcoholic fatty liver, Hepatology, 43, 989-1000
Hanus L, Breuer A, Tchilibon S, Shiloah S, Goldenberg D, Horowitz M, Pertwee R G, Ross R A, Mechoulam R, Fride E. HU-308: a specific agonist for CB(2), a peripheral cannabinoid receptor. Proc Natl Acad Sci USA. 1999 Dec. 7; 96(25):14228-33.
Harlow E. et al., Antibodies: A Laboratory Manual, Cold Spring Harbor Laboratory, New York, (1988).
Huffman J W, Liddle J, Yu S, Aung M M, Abood M E, Wiley J L, Martin B R. 3-(1′,1′-Dimethylbutyl)-1-deoxy-delta8-THC and related compounds: synthesis of selective ligands for the CB2 receptor. Bioorg Med. Chem. 1999 December; 7(12):2905-14.
Julien B, Grenard P, Teixeira-Clerc F, Van Nhieu J T, Li L, Karsak M, Zimmer A, Mallat A, Lotersztajn S. Antifibrogenic role of the cannabinoid receptor CB2 in the liver. Gastroenterology. 2005 March; 128(3):742-55.
Köhler G, Milstein C. “Continuous cultures of fused cells secreting antibody of predefined specificity”. Nature. 1975; 256(5517):495-7.
Kwoh et al., 1989, Proc. Natl. Acad. Sci. USA, vol. 86, p: 1173-1177, 1989.
Lizardi et al., Biol. Technology, vol. 6, p: 1197, 1988.
Matsuda et al., 1990 Nature (Lond) 346: 561-564.
Parfieniuk A, Flisiak R. Role of cannabinoids in chronic liver diseases. World J. Gastroenterol. 2008 Oct. 28; 14(40):6109-14.
Pertwee R G. Pharmacology of cannabinoid receptor ligands. Curr Med. Chem. 1999 August; 6(8):635-64.
Rinaldi-Carmona M, Calandra B, Shire D, Bouaboula M, (justric D, Barth F, Casellas P, Ferrara P, Le Fur G. Characterization of two cloned human CB1 cannabinoid receptor isoforms. J Pharmacol Exp Ther. 1996 August; 278(2):871-8.
Rinaldi-Carmona et al., J. Pharmacol. Expt. Ther., 1998, 284:644-650.
Teixeira-Clerc F, Julien B, Grenard P, Tran Van Nhieu J, Deveaux V, Li L, Serriere-Lanneau V, Ledent C, Mallat A, Lotersztajn S. CB1 cannabinoid receptor antagonism: a new strategy for the treatment of liver fibrosis. Nat. Med. 2006 June; 12(6):671-6.

1-5. (canceled)

6. A method of preventing or treating alcoholic liver disease in a subject, comprising the step of
administering to said subject a selective CB2 agonist.

7. The method of claim 6, wherein said selective CB2 agonist is [(6aR,10aR)-3-(1,1-dimethylbutyl)-6a,7,10,10a-tetrahydro-6,6,9-trimethyl-6Hdibenzo[b,d]pyran] (JWH-133).

8. A method of preventing or treating alcoholic hepatic steatosis in a subject, comprising the step of
administering to said subject a selective CB2 agonist.

9. The method of claim 8, wherein said selective CB2 agonist is [(6aR,10aR)-3-(1,1-dimethylbutyl)-6a,7,10,10a-tetrahydro-6,6,9-trimethyl-6Hdibenzo[b,d]pyran] (JWH-133).
